(19) United States
(12) Patent Application Publication
(10) Pub. No.: US 2008/0097526 A1
(43) Pub. Date: Apr. 24, 2008

(54) Surgery procedure for mastopexy

(75) Inventor: Ciro Accardo, Portici (IT)

Correspondence Address: YOUNG & THOMPSON, 745 SOUTH 23RD STREET, 2ND FLOOR, ARLINGTON, VA, 22202, US

(73) Assignee: PROMOITALIA INTERNATIONAL SRL, Napoli (IT)

(21) Appl. No.: 11/907,437

(22) Filed: Oct. 12, 2007

(30) Foreign Application Priority Data
Oct. 12, 2006 (IT) RM2006A000550

(51) Int. Cl. A61B 17/04; A61B 17/08; A61D 1/00

(52) U.S. Cl. 606/228; 606/215; 606/216

(57) ABSTRACT

Mastopexy or breast lift surgery involves inserting permanent and reabsorbable threads under the skin that follow a skin marker drawn by the operator. This enables the interested area to be lifted without leaving vertical or inverted T scars on the patient's breast or any other type of scar.

### FIELD OF THE INVENTION

This idea consists of an innovative technique for breast or mastopexy surgery.

### BACKGROUND OF THE INVENTION

Mastopexy allows lifting a sagging breast. Excessive weight loss, pregnancy, breastfeeding, force of gravity and normal ageing process are manifested through the clinical condition known as dermatochalasis (ptosis) of soft tissues surrounding the mammary gland and the gland itself.

This involves the progressive relaxation of the connective tissues that envelop the gland and the intra-glandular stremal tissues. The result is mammary ptosis which causes aesthetic problems for many women.

In respect to traditional mastopexy techniques which shape the breast by bringing the form back to its previous harmony through the removal of a rather large portion of the lower quadrants of the mammary gland, this new mastopexy technique produces this same effect without amputating any part of the gland and without having to transfer the areola-nipple complex. Therefore, no visible scars are left on the patient's skin.

Many techniques currently exist for lifting the breast: the choice depends on the specific clinical conditions of the patient.

Not taking into account the technical details, the differences between the various operating procedures are often translated into a different extension of the remaining scars.

The continual search for new reduction and mastopexy methods focuses on reducing to a minimum scars which inevitably exist after an operation.

### SUMMARY OF THE INVENTION

This idea aims at providing a technique for mastopexy procedures based on inserting permanent and/or reabsorbable threads under the skin that follow skin paths marked by the operator for lifting the interested area.

This solution has numerous advantages. In fact, unlike tradition techniques:

A) there is no need to remove part or sections of the mammary gland or to transfer (i.e. move) the areola;

B) no vertical or inverted T scars remain on the breast, armpit or any other part of the body;

C) in comparison, there are fewer complications;

D) there are no recovery times, the patient remains in a clinic or day hospital for 2-4 hours.

The correct clinical indication is for patients with breast sizes between a B and D cup and with enough breast volume to cement the breast lift. The patient must also have a sufficient quantity of subcutaneous intra-areola fat in the mammary gland.

This procedure is not suitable for large breasts (cup sizes over D and a distance of over 26 cm from the middle III of the clavicle to the upper nipple) or for breasts without the necessary fat components or with too much excess skin. Traditional procedures and techniques are required in these cases.

The ideal age bracket is between 27 and 45 years. Clinical conditions permitting, this technique can also be applied to older patients.

### BRIEF DESCRIPTION OF THE DRAWINGS

A better understanding of the idea can be obtained from the detailed description that follows, referring to the attached drawings and photos that illustrate the various phases of the Mastopexy technique with suspended threads. The figures:

FIGS. 1, 2, 3 and 4 show the operations for identifying the three periosteal anchors;

FIGS. 5 and 6 show the dividing of the breast into four quadrants;

FIGS. 7, 8, 9, 10 and 11 show the outline and definition of the crease below the breast and the radial courses that go from the lower areolar margin to the breast crease, and the net of threads in the lower quadrants;

FIGS. 12 and 13 show the drop-shaped path for lifting the area from the areola region to the breast;

FIGS. 14 to 17 show the marking procedure of the thread path if the nipple needs lifting. This requires the use of reabsorbable threads;

FIGS. 18 and 19 show the path of a second reabsorbable thread, once again for lifting the nipple, if necessary;

FIGS. 20, 21, 22 and 23 show the path of the permanent threads that follow a course parallel to the crease below the breast which are the same distance apart;

FIG. 24 is an enlarged view of the start and end points of the paths illustrated in FIGS. 20 to 23;

FIGS. 25 and 26 show the marked paths of the permanent threads which are concentric to the mammary sagittal axis in the case of two and three threads respectively;

FIGS. 27, 28 29 and 30 show the different phases of the local anaesthesia;

FIG. 31 shows the sterilisation of the skin for creating a sterile operating area;

FIGS. 32, 33 and 34 show the operator making the periosteal anchors;

FIGS. 35, 36, 37 and 38 show the various phases of inserting the permanent and reabsorbable threads in correspondence of their subcutaneous courses for creating a net of threads that interweaves inside the two lower quadrants of the breast;

FIGS. 39 and 40 show the introduction of another permanent thread with a course in the shape of an elongated drop to create the lifting of the alveolar region.

FIG. 41 relates to the lifting of the nipple through the use of two reabsorbable threads with different diameters;

FIGS. 42 and 43 show the various phases of applying the bandage;

FIG. 44 show the brace to use for a certain period after the removal of the bandage.

### DETAILED DESCRIPTION OF THE INVENTION

With reference to the figures, we will now look at each phase of the mastopexy technique according to the invention.

I) Marker of Skin Paths

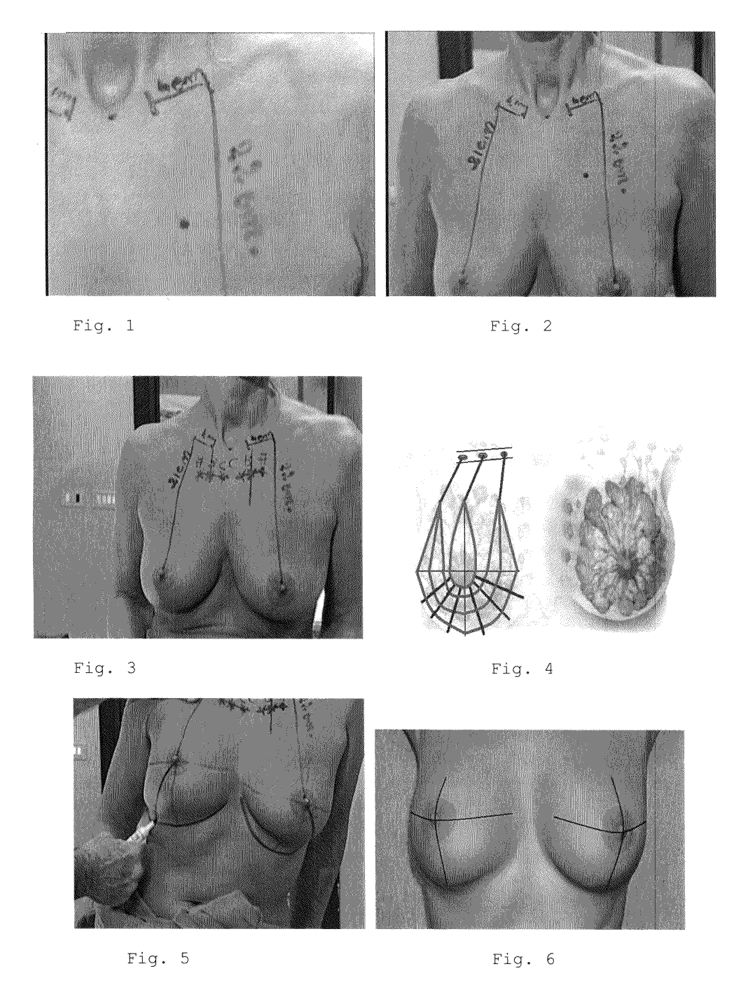

Using a permanent marker, the operator must identify the right and left distal extremity of the clavicle and, using a decimetre, take off a point at a distance of 4 cm from the clavicular sternum articulation (FIG. 1).

The distance from the homolateral nipple will be measured from this point located on the upper margin of the clavicle. This distance must be less than 26 cm in order for this technique to be implemented (FIGS. 2 and 3).

After this, the second rib and its sternal-rib insertion will be measured with precision.

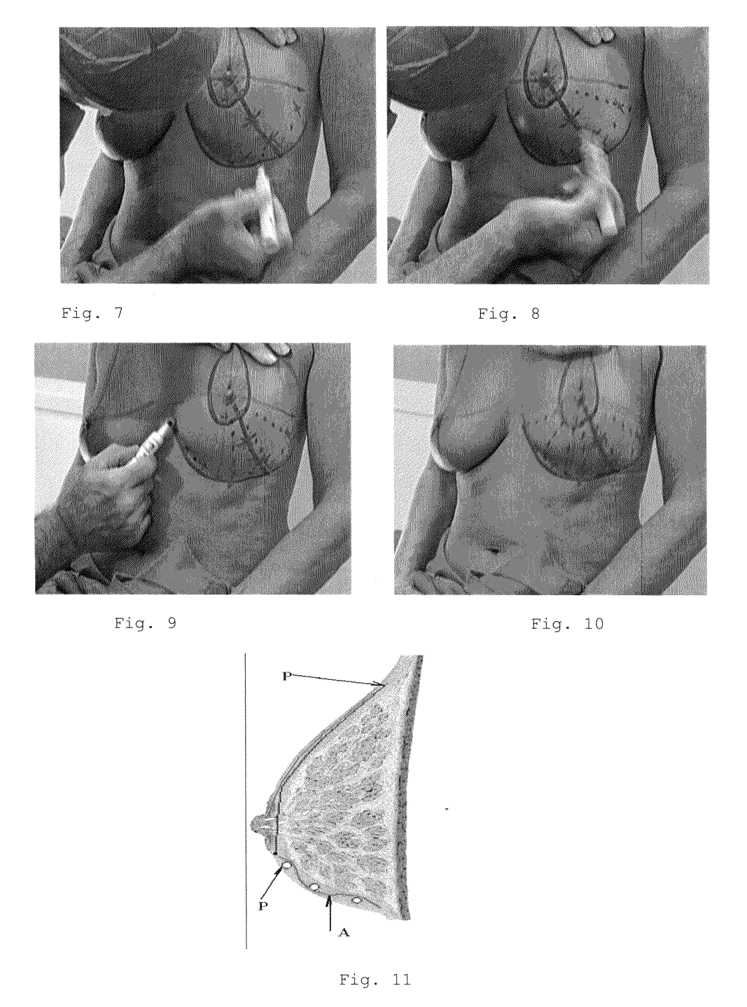

At this point, three points will be defined on the skin surface (FIG. 4) which will correspond in projection to the three anchoring points on the periosteums of the front face of the second rib. Using a scalpel with a 2 mm blade, three small incisions are made next to these points, or alternatively, the incisions can be made with a derma-punch having a diameter of 2.2 mm.

These three points are located on the front face of the second rib next to its middle point. The first point is located at around 2 cm from the costal-sternal articulation, the second point at around 2½-3 cm moving sideways and the third at 2½-3 cm even further sideways (the variability of the distance relating to the three anchors will depend on the larger or smaller size of the patient's thoracic cage).

These three points identify the three periosteum anchors.

The same thing is carried out on the contra-lateral rib.

The operator must then divide—always with the help of a permanent marker—the breast into four quadrants according to the two orthogonal axes passing through the nipple (vertical axis, horizontal axis). Thus dividing the mammary gland into four quadrants (FIGS. 5 and 6):

A) Internal upper quadrant

B) External upper quadrant

C) Internal lower quadrant

D) External lower quadrant

After executing this operation, the crease below the breast is outlined and identified with a skin marker.

The external lower quadrant is then divided by an oblique line which starts from the nipple and goes to the crease below the breast forming a 20-30 degree angle (measured from the vertical axis and moving in a clockwise direction). The segment marked on this line—which has one extremity on the areolar margin and the other extremity next to the crease below the breast—must be divided into three or four equal segments depending on what will be inserted into the lower quadrants of the breast: two or three 0/2 USP calibre barbed threads with the trade name of “Breast Up Threads Happy Lift™ Revitalizing registered with U.S. Pat. No. ______.

Six, nine or twelve radial paths are drawn in the two lower quadrants that depart from the lower areolar margin and end in correspondence of the breast crease.

Inserting a total of six threads: three Happy Lift™ Revitalizing and reabsorbable threads with 2/0 USP calibre radiating and converging on the lower areolar margin must be inserted into each of the two lower quadrants. They must be positioned according to the axes at angles of 15°, 45°, 75°, 105°, 135°, 165° and move in a clockwise direction departing from the horizontal axis of the breast.

Instead, inserting nine threads into the two quadrants, the angular distance between the six threads arranged in a radial manner in the two lower quadrants, converging on the lower areolar margin, will be the same as that described in the previous paragraph for the internal threads of the two lower quadrants.

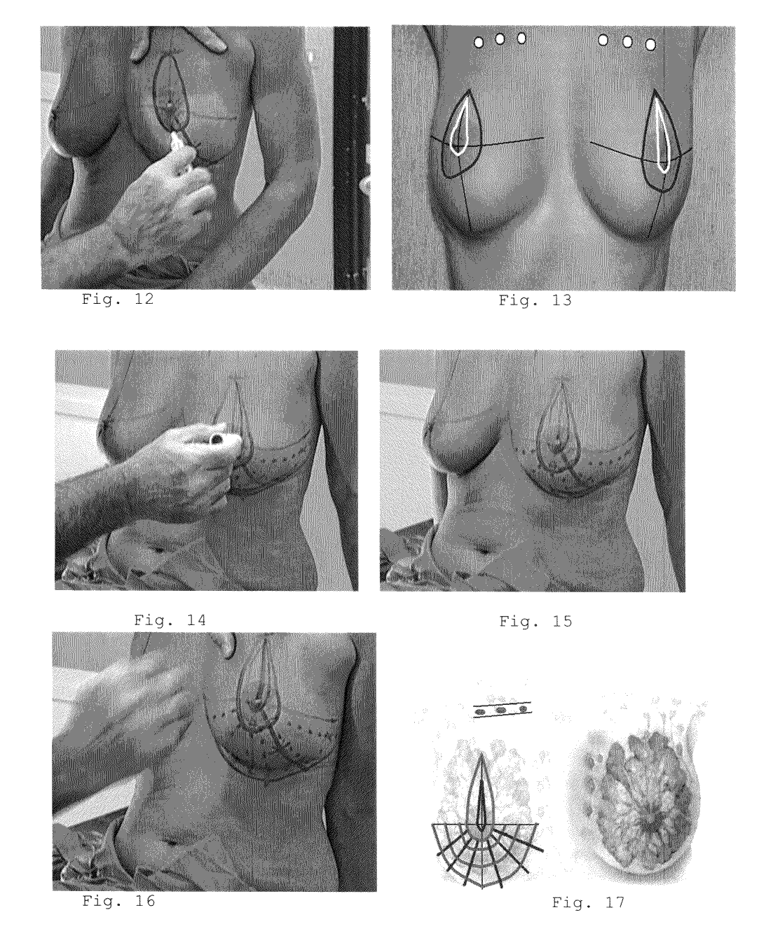

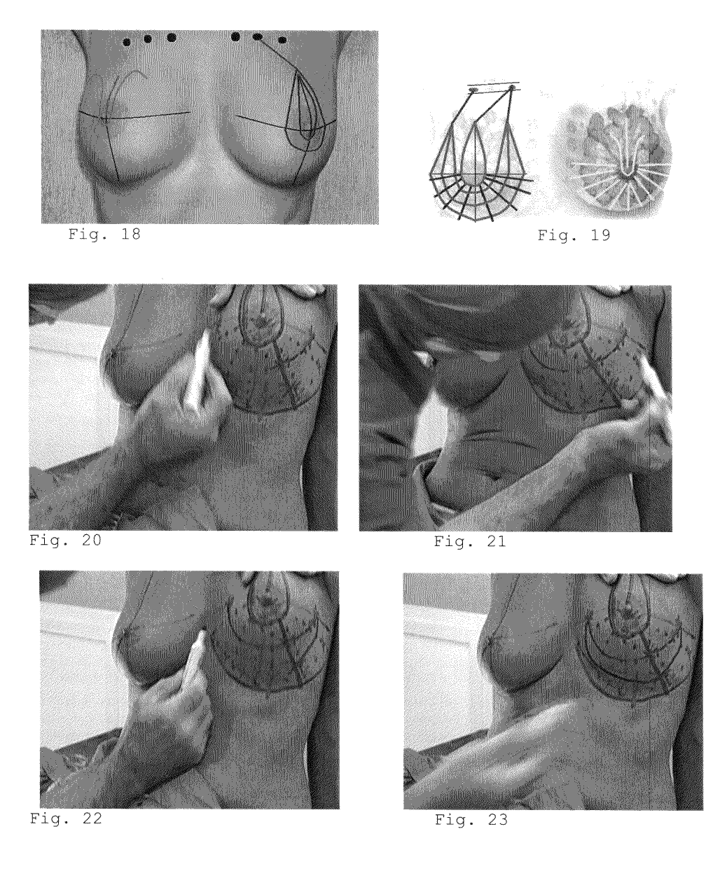

The other three threads will be arranged in this order: one on the vertical axis and the other two on the horizontal axis in a centrifugal direction from the areolar margin to the crease below the breast (FIGS. 7, 8, 9 and 10).

If eleven radial threads are introduced, the angular distance between each thread will be around 22.5°, including the vertical thread and the two horizontal threads.

At this point, a line for lifting the areolar region of the breast will be marked. The course of this line will be shaped like a drop and the apex will be located on the vertical axis of the breast at around 7 cm from the upper margin of the areola (FIGS. 12 and 13).

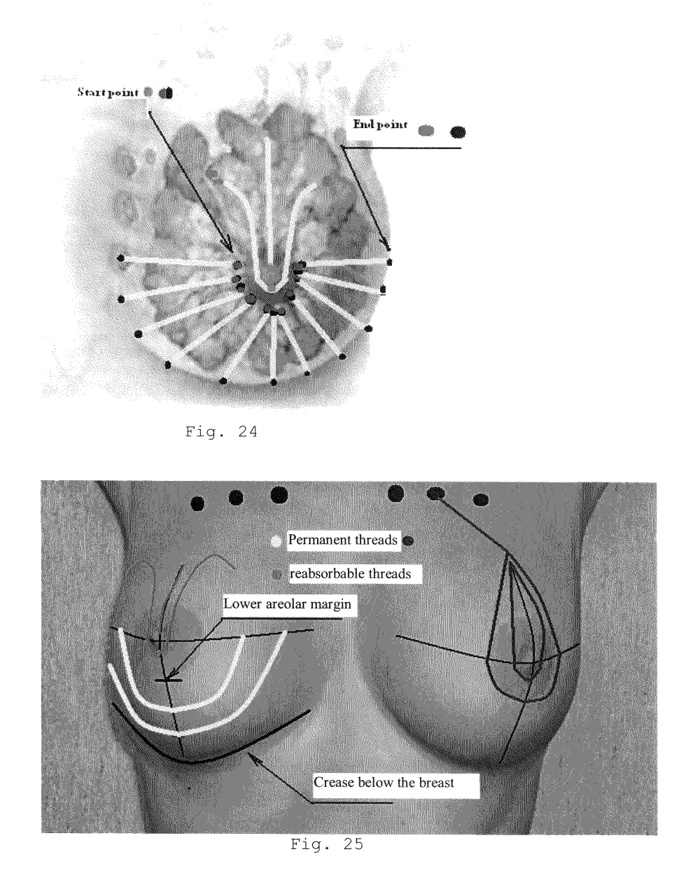

If the nipple requires lifting, the path of the thread must be marked (FIGS. 14, 15, 16 and 17). Reabsorbable threads will be used for this procedure.

Depending on the case, one or two threads may be used. One of the two threads used (the 12 cm long 2/0 USP calibre Happy Lift™ Revitalizing thread) will have a course that will first follow the vertical upper margin of the nipple and then, after making a turn of 90°, move vertically along the vertical axis of the breast until emerging 6-7 cm from the upper margin of the areola.

If a second 23 cm thread needs to be inserted, then its course will encircle the lower margin of the nipple and move upwards at an angle of 25-300 in respect to the vertical axis for both the right and left side. It will end in a hook-shaped course whose peak will be located at around 7-8 cm in respect to a horizontal plane tangent to the upper margin of the areola (FIGS. 18 and 19).

Finally, the course of the permanent threads must be defined. This consists of concentric paths at the crease below the breast which are made in such a way that they are the same distance apart (FIGS. 20, 21, 22 and 23). Therefore, if only one thread is inserted, it will pass through the middle point of the vertical axis and through the middle points of the two horizontal semi-axes.

The start and end points of these paths are equidistant and found on horizontal segments located respectively to the right and left of the horizontal axis which is centred on the nipple. They move in a radial direction from the areolar margin to the breast crease (FIG. 24).

If positioning two permanent 0/2 USP calibre Breast Up Happy Lift™ Revitalizing threads, they will be located in an equidistant manner on the previously created segment with a circular concentric course in respect to the sagittal axis of the breast (FIG. 25).

Likewise, if three threads are positioned instead of two, they will be positioned in a concentric manner in respect to the sagittal axis of the breast, only that in this case the radial segment running from the areolar margin to the breast crease will now be divided into three or four equal parts (FIG. 26).

These two or three threads will then be made to flow into and emerge from a point at around 3-4 cm on the vertical axis which divides the right and left horizontal semi-axis into two equal parts.

At this point, the operation can proceed.

II) Anaesthesia

Introduction:

A central vein of the patient will need to be located which must also be available for any further sedation if the patient has very low analgesic sensitivity.

The patient must be constantly monitored and an anesthetist must always be present during the operation, which must be carried out in an operating room.

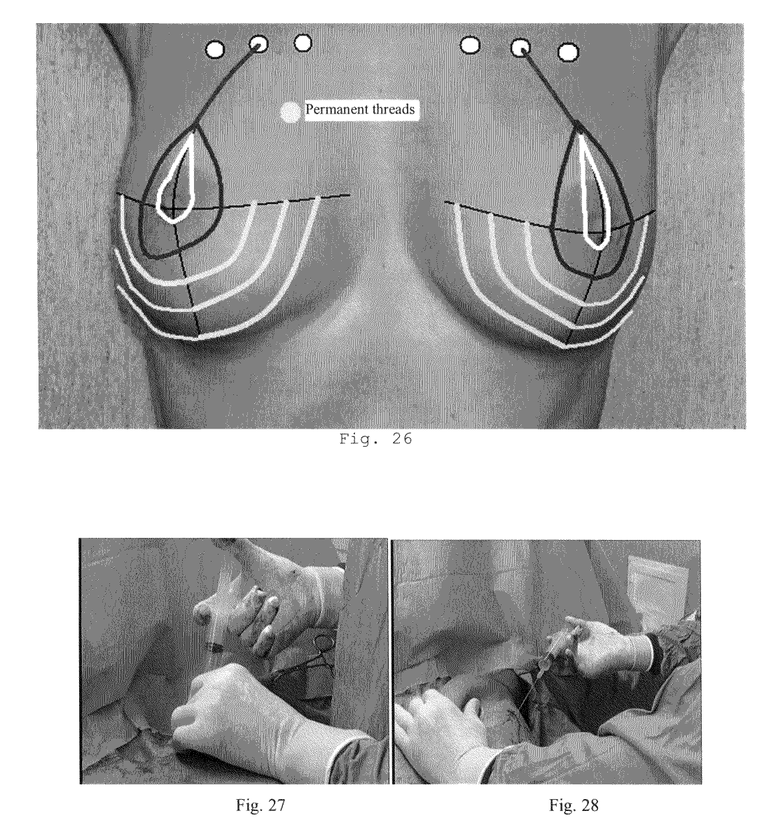

For this type of operation, a mild anaesthetic premeditation is required with supervision of the patient. This will be followed by a local anaesthetic being injected into the entry and exit holes of the hooked needle with hole or the cannula. After this phase, the indicated paths are anaesthetized as shown in FIGS. 27, 28, 29 and 30.

An anaesthetic on the costal periosteum will also be carried out at the level of the second or third rib in correspondence of its front face and, more precisely, in its middle point next to the two or three periosteal anchoring points.

Particularly sensitive patients must assume benzodiazepine derivatives or other non-hypnotic sedatives orally at least one hour before the operation.

III) Anchoring to the Periosteum

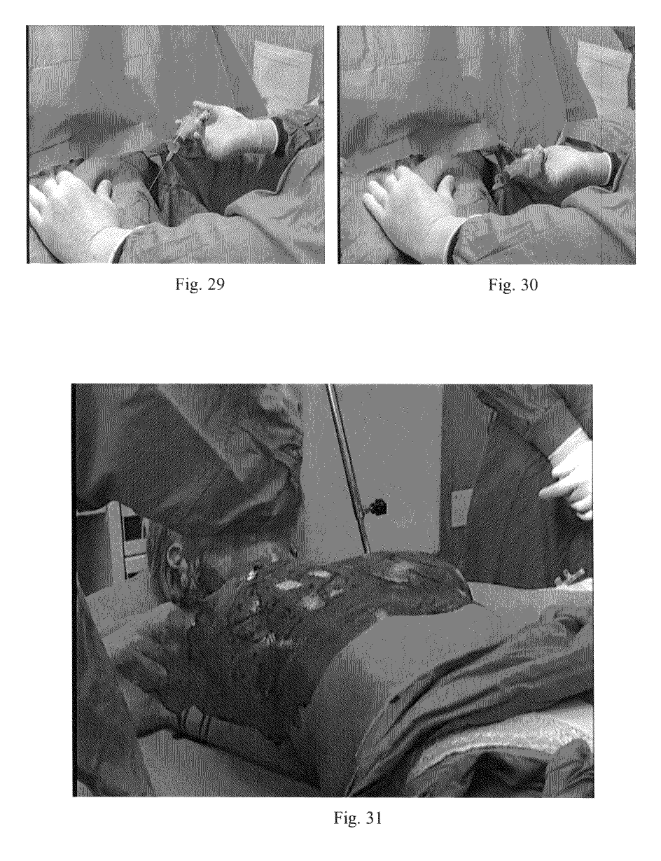

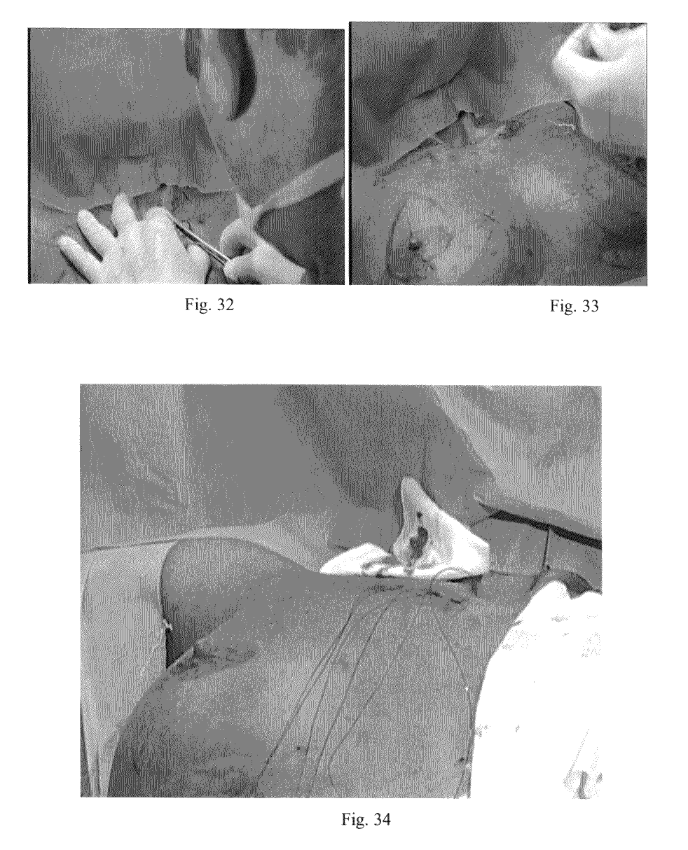

The next operating phase begins after the skin has been carefully sterilised so that an absolutely sterile operating field is created (FIG. 31).

At this point, the operator shall make two or three periosteum anchors. The number varies depending on the size of the patient's breast (FIGS. 32 and 33).

Two or three anchors are made as follows: the first anchor is positioned at around 2½-3 cm from the sternal rib articulation of the second or third rib, the second at around 2½ cm from the first and the same division if a third anchor is necessary.

This is the most delicate phase of the entire operation because of the difficulty in anchoring the USP 2/0 or 0/0 or 1/0 prolene thread onto the costal periosteum and because this anchor must be made in correspondence of the middle point of the front face of the rib at an equal distance from the upper and lower margin.

It is important to not get too close to the lower costal margin since it could cause lesions to the vessels and costal nerves located below this margin.

Furthermore, the operator must be absolutely sure to have the needle between the index and middle finger of the left hand (FOR RIGHT-HANDED OPERATORS) when introducing the thread into the upper and lower margins of the second rib. The needle must be introduced between these two fingers in order to not produce an iatrogenic pneumothorax.

Introduce the thread which has been placed on a cylindrical needle (½ c. cyl. 76 mm USP 2/0) and pass it deeply below the costal periosteum for a length of around ½ cm. Let the cylindrical needle with atraumatic needle exit at around 2-2½ cm from the entry hole along the transversal axis of the middle rib.

The second return passage will be on the surface and will start at the exit hole towards the initial hole. After pulling the thread and making sure it has hooked onto the periosteum, a running knot will be made at the end to securely anchor the thread. The needle is then cut from the extremity and the two free extremities are clamped with a small fastener (this thread will then be used to anchor the permanent threads after having transferred them under the skin in correspondence of the anchoring points—see figure—the extremities of the permanent threads encircling the lower quadrants of the breast and the thread encircling the perimeter of the areola). The same thing occurs for the second and third periosteal anchors (See FIG. 34).

IV) Insertion of the Permanent and Reabsorbable Threads

After creating six or four periosteal anchors bilaterally, the reabsorbable and permanent threads are inserted in correspondence of their subcutaneous paths.

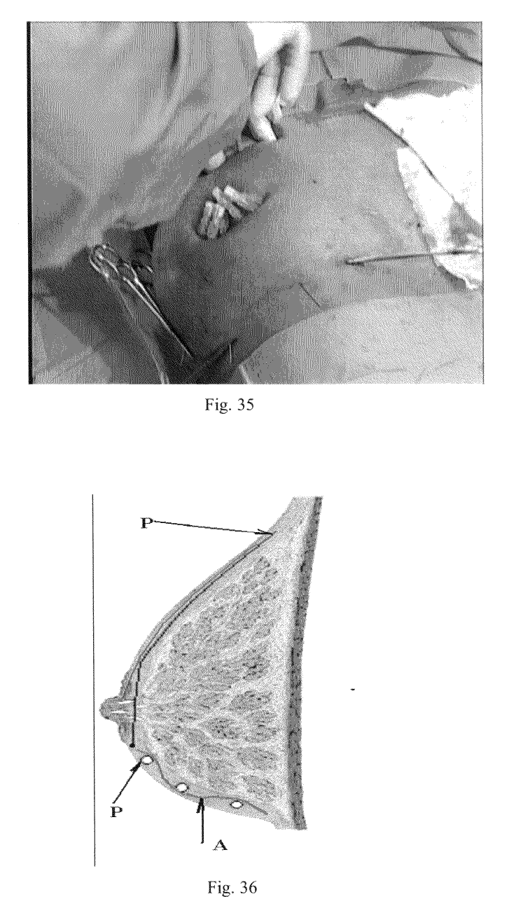

Six or nine cannulas (120 mm 20 G/0.90 mm) will be introduced—depending on the volume and size of the implant base of the breast (FIG. 35).

Following the markers previously made on the skin surface, the cannulas are inserted in a radial direction or from the areolar periphery towards the breast crease.

The cannula needle or curvilinear needle with hole will then be introduced for implanting the permanent 0/2 USP calibre Breast Up Happy Lift™ Revitalizing threads.

The cannula with spindle or needle with hole are introduced by moving them forward in a sinusoidal direction, or alternatively, once on top of the cannula arranged in a radial manner and then below the next cannula and along the entire lower quadrant (first in the internal lower quadrant and then in the external lower quadrant). The same procedure occurs if a second thread or third permanent 0/2 USP calibre thread is introduced.

In this way, a net is created with the threads which interweaves inside the two lower quadrants of the breast (FIGS. 36, 37 and 38).

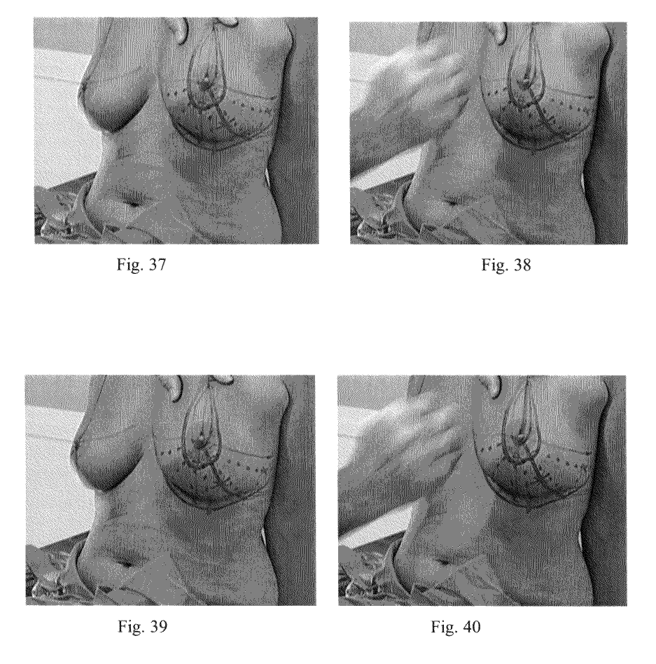

Depending on the dimensions and size of the breast, two or three permanent threads are inserted into the two lower quadrants according to a course that runs concentric to the nipple of the breast (see FIGS. 37 and 38).

The extremities of the Breast Up Happy Lift™ Revitalising barbed threads are then introduced into the extractor (if a cannula is used) or hole (if a hooked needle is used) after the hooked needle with hole has been extracted or after the extractor has been pulled from the distal hole of the cannula.

After these manoeuvres, the two extremities of the prolene thread will emerge from the two holes—start and end points for lifting the lower breast region.

After implanting one, two or three prolene threads the reabsorbable Happy Lift™Revitalizing 2/0 USP calibre threads are introduced into the cannulas arranged in a radial manner, keeping the skin clamped with the index finger and thumb in correspondence of the entry and exit holes.

The cannulas are extracted and the threads will remain lodged in the hypodermic plane intersecting with the permanent threads introduced beforehand.

Around 1-1½ cm of the free extremities of the reabsorbable threads will emerge. At the end of the operation, these extremities will be secured with Steri-strips on the areolar surface or on the surface next to the crease below the breast. The bandage and plastering of the mammary glands realised with 2 cm-wide paperboard or micropore paper tape are removed on the second or third day and the extremities are cut 2 mm below the skin surface. Another permanent thread with an elongated drop-shaped course (see FIGS. 39 and 40) can be introduced which will encircle the lower perimeter of the areola for lifting the areolar region.

This thread must be introduced in a point located at around 7 to 8 cm on the vertical axis of the breast using a needle with hole or cannula. It must move in a cranial-caudal direction and exit at the point where the mammary axis intersects the lower circumference of the areola.

Half the extremity of the prolene thread will be inserted into the hole and extractor of the needle-cannula, making sure that the middle point of the thread is positioned in correspondence of the point where the mammary axis intersects the lower areolar margin.

This same procedure will be repeated on the left hemi-course under the skin departing from the start point and exiting at the end point, which is located in the aforementioned intersection point.

The remaining part of the other extremity of the prolene thread will be introduced into the hole of the needle or into the extractor of the cannula and will also be made to exit from the start point.

V) Lifting of the Areola and Nipple

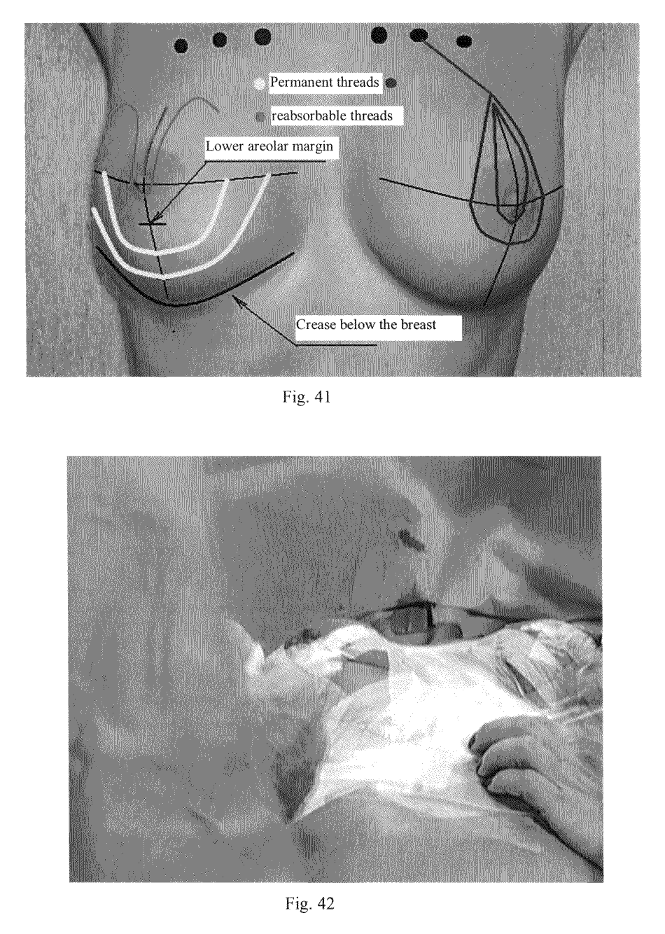

Two reabsorbable threads are required for lifting the nipple. The first thread must be 12 cm long and the other 23 cm. The 23 cm thread must be positioned with a course that encircles the bottom of the lower perimeter of the nipple (see FIG. 41). It must then move upwards from both sides in a curvilinear “S” movement and end with a hooked course, as described above.

Once again, the middle point of the thread must be positioned in correspondence of the point where the mammary axis intersects the lower margin of the nipple.

The needle will be introduced from the upper distal extremity of the course and made to exit at the intersection point mentioned.

The extractor of the thread is introduced into the hole. Half of the thread is inserted into the subcutaneous tissue while the other half is introduced using the same technique, that is, by introducing the cannula into the other upper proximal hole.

As for the 12 cm thread, it will be introduced as simply as possible proceeding from the upper extremity of the nipple—i.e. the point where the vertical axis of the breast intersects the upper margin of the nipple—and continue vertically for the entire thickness of the nipple until reaching the areola. In this point, the needle is turned at an angle of 90° so that it is parallel to the skin surface that covers the areola and breast. It moves forward into the hypodermic plane in a caudal/cranial direction and exits along the mammary vertical axis at around 6 cm from the upper margin of the areola.

The 12 cm thread is then introduced. Its middle point must correspond to the middle point of the course and, after the extraction of the cannula needle, the thread remains in place.

This technique using two reabsorbable threads is also for lifting and correctly positioning the nipple.

VI) Bandaging

Once the operation has been completed, the breasts must be bandaged for 48 to 72 hours (FIG. 42).

This bandaging must be carried out using elastic bandages with a thickness of 8-10 cm. They must not be too tight so that ischemic damage is not caused to the operated areas.

First, wrap sticking plaster (micropore or transpore) around the two breasts. 10-20 cm sections are taken from the support spool and applied on the breasts so that they are suspended and brought together in the correct position.

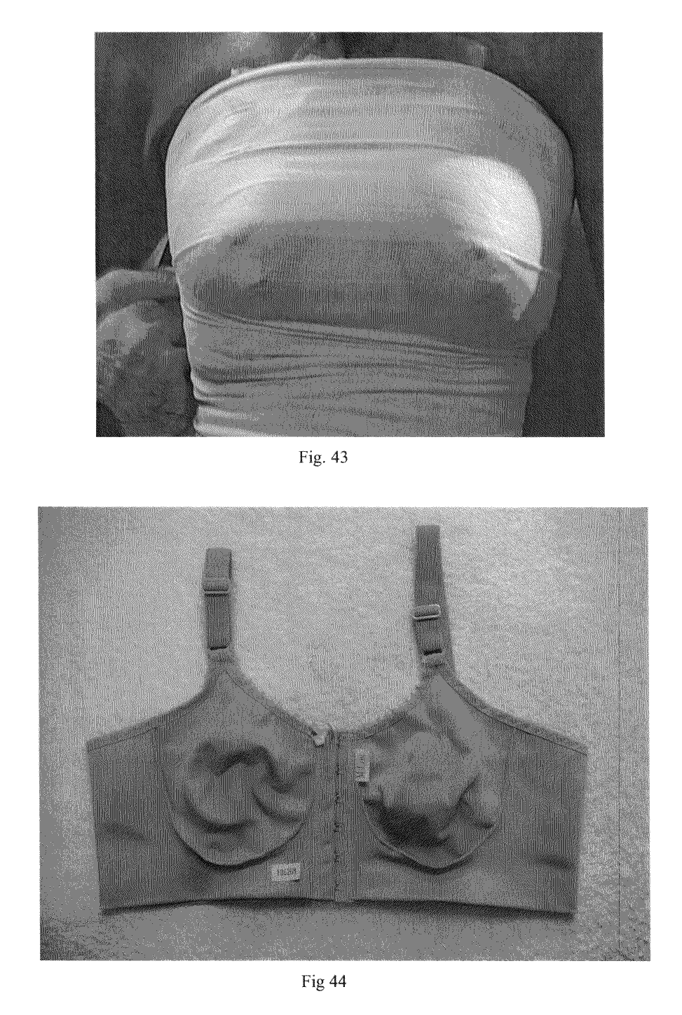

After plastering the two breasts, an elastic bandage with a thickness of 12 cm is applied to create a corset for the bust (the bandage is not necessary and may be replaced by an elastic brace) (see FIG. 43).

After two days, this bandage can be carefully removed by the operating surgeon. After the extremities of the reabsorbable radial threads are cut and the skin is sterilised, it can be replaced with a brace for the bust that the patient must wear for 45-60 days without ever removing it. This brace may also be worn in the shower since it is made of nylon (FIG. 44)

1. Surgical procedure for breast lifts characterised by the fact that permanent and reabsorbable threads are inserted under the skin which follow skin paths marked by the operator to lift the interested region, without leaving vertical scars or inverted T scars on the patient's breasts or scars in the armpit area.

2. Surgical procedure for breast lifts according to claim 1, characterised by the fact that the skin paths are marked as follows:
a) three periosteal anchoring points are identified;
b) with the help of a permanent marker, the breast is divided into four quadrants along the two orthogonal axes passing through the nipple (vertical axis, horizontal axis): internal upper, external upper, internal lower, external lower;
c) the crease below the breast is outlined and marked with a skin marker;
d) the external lower quadrant is divided via an oblique line that, departing from the nipple, reaches the crease below the breast forming an angle of 25-30° (measured in a clockwise direction from the vertical axis); the segment outlined on this line—which has one extremity on the areolar margin and the other in correspondence of the crease below the breast—being divided into three or four equal segments depending on whether two or three permanent or reabsorbable 0/2 USP calibre barbed threads are inserted into the lower quadrants of the breast;
e) six or nine radial courses are drawn in the two lower quadrants which depart from the lower areolar margin and terminate in correspondence of the mammary crease, and
f) a drop-shaped course is drawn for lifting the areolar region of the breast, which peaks at the vertical axis of the breast located at around 7-8 cm from the upper margin of the areola.

3. Surgical procedure for breast lifts according to claim 1, characterised by the fact that a total of six-nine-eleven threads are inserted in phase (e); three 2/0 USP calibre reabsorbable threads are inserted into each of the two lower quadrants arranged in a radial manner and converging on the lower areolar margin located according to the axes in a 15°, 45°, 75°, 105°, 135°, 165° direction (there are six or nine threads if we also cover the axes, the horizontal semi-axes and the lower vertical semi-axes) 11.25°, 33.75°, 56.25°, 78.75°, 101.25°, 123.75°, 146.25°, 168.75° (if there are eleven threads) and moving in a clockwise direction departing from the horizontal axis of the breast.

4. Surgical procedure for breast lifts according to claim 2, characterised by the fact that if nine threads are inserted into the two quadrants in phase (e), the six threads arranged in a radial manner in the two lower quadrants which converge on the lower areolar margin, are located according to the axes in a 15°, 45°, 75°, 105°, 135°, 165° direction and move in a clockwise direction departing from the horizontal axes of the breast while the other three threads are arranged as follows: one on the vertical axis and the other two on the horizontal axis moving in an outward direction from the centre of the areolar margin to the crease below the breast.

5. Surgical procedure for breast lifts according to claim 2, characterised by the fact that the following procedures must first be carried out if the nipple needs to be lifted:
the course for one or two reabsorbable threads must be marked; the first thread must follow a path that first goes from the vertical upper margin of the nipple and then, after making a 90° turn, must move vertically along the vertical axis of the breast until emerging 7-8 cm from the upper margin of the areola; the second thread, must encircle the lower margin of the nipple and move upwards in a vertical direction at around 1½ cm from the vertical axis of the breast until emerging with a hooked path at around 7-8 cm from the upper margin of the areola; and
the path of the permanent threads that follow a course parallel to the crease below the breast must be marked and realised in such a way that there is an equal distance between each thread; since the start and end points of these paths are equidistant and found on horizontal segments located to the right and left of the horizontal axes which are centred on the nipple, they move radically from the areolar margin to the breast crease.

6. Surgical procedure for breast lifts according to claim 1, characterised by the fact that the operating phase requires two or three periosteal anchors to be made depending on the size of the patient's breast; the first anchor is made at around 2½-4½ cm from the costal-sternal articulation of the second or third rib, and the second at around 3-4 cm from the first and the same for a third anchor, if necessary.

7. Surgical procedure for breast lifts according to claim 6, characterised by the fact that the thread on the costal periosteum is anchored in correspondence of the middle point of the front face of the rib with an equal distance between the upper and lower margin.

8. Surgical procedure for breast lifts according to claim 6, characterised by the fact that the anchoring of the thread requires:
introducing the thread mounted on a cylindrical needle (½ c. cyl. 76 mm USP 2/0) and passing it deep below the costal periosteum for a length of around ½ cm and making the cylindrical needle with atraumatic needle exit at around 3-4 cm from the entry hole along the transversal axis of the middle rib;
a second surface return passage to be made, which starts at the exit hole and exits in correspondence of the first incision;
after having pulled the thread and making sure it hooked onto the periosteum, a running knot must be made at the end to secure the thread deep down;
cutting the needle from the extremity and clamping the two free extremities with a small fastener so that this thread can then be used to anchor the permanent threads after transferring the extremities of the permanent threads encircling the lower quadrants of the breast and the thread encircling the areolar perimeter under the skin in correspondence of the anchoring points;
proceed in a similar way for the second and third periosteal anchors.

9. Surgical procedure for breast lifts according to claim 1, characterised by the fact that once the periosteal anchors have been made, the reabsorbable and permanent threads are then inserted in correspondence of their subcutaneous paths; a variable number of cannulas (120 mm 20 G/0.90 mm) are introduced—depending on the volume and size of the implant base of the breast—which follow the marks previously made on the skin surface; these cannulas being inserted in a radial direction or in the direction of the areolar periphery towards the breast crease and a cannula needle or curvilinear needle with hole is introduced for implanting the permanent threads.

10. Surgical procedure for breast lifts according to claim 9, characterised by the fact that these permanent threads are 0/2 USP calibre permanent threads marketed under the name of Breast Up Happy Lift™ Revitalizing.

11. Surgical procedure for breast lifts according to claim 10, characterised by the fact that the cannula with spindle or needle with hole are introduced by moving them forward in a sinusoidal direction, or alternatively, once on top of the cannula arranged in a radial manner, and then below the next cannula along the entire lower quadrant (first in the internal lower quadrant and then in the external lower quadrant); the same procedure occurring if a second or third 0/2 USP calibre permanent thread is introduced so that a net of threads is created which interweaves inside the two lower quadrants of the breast.

12. Surgical procedure for breast lifts according to claim 11, characterised by the fact that the extremities of the permanent barbed threads are introduced into the extractor (if using a cannula) or into the hole (if using a hooked needle) after having extracted the hooked needle with hole or after having made the extractor exit from the distal hole of the cannula, so that the two extremities of the prolene thread emerge from the two holes—start and end points—required for lifting the lower breast region.

13. Surgical procedures for breast lifts according to claim 12, characterised by the fact that after implanting two or three prolene threads, 2/0 USP calibre reabsorbable threads are introduced into the cannulas arranged in a radial manner, keeping the skin clamped with the index finger and thumb in correspondence of the entry and exit holes; the cannulas are then extracted and the threads remain lodged in the hypodermic plan to intersect with the previously introduced permanent threads.

14. Surgical procedure for breast lifts according to claim 5, characterised by the fact that two reabsorbable threads are used for lifting the nipple: the first is 12 cm long and the second is 23 cm long; the 23 cm thread is positioned according to a course that encircles the lower perimeter of the nipple at the bottom and then move upwards from both sides in a “S” shaped curvilinear course ending with a hooked path; the aim is to position the middle point of the thread in correspondence of the point where the vertical axis of the breast intersects the lower margin of the nipple.

15. Surgical procedure for breast lifts according to claim 14, characterised by the fact that the needle is introduced starting at the upper distal extremity of the path and ending at the intersection point described above.

16. Surgical procedure for breast lifts according to claim 14, characterised by the fact that the 12 cm threads are introduced starting from the upper extremity of the nipple—in the point where the vertical axis of the breast intersects the upper margin of the nipple—and continue vertically for the entire thickness of the nipple until arriving at the areola where the needle is turned at a 90° angle so that it is parallel to the skin surface; it moves forward in a caudal-cranial direction into the hypodermic plan and made to exit at around 6 cm from the upper margin of the areola along the vertical axis of the breast.
